(19) United States
(12) Patent Application Publication
(10) Pub. No.: US 2005/0164041 A1
(43) Pub. Date: Jul. 28, 2005

(54) Medical device having a smooth, hardened surface

(76) Inventors: David V. Dunsmore, Tualatin, OR (US); Hubert Patrovsky, Laval (CA)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 10/768,762

(22) Filed: Jan. 29, 2004

Related U.S. Application Data
(60) Provisional application No. 60/538,551, filed on Jan. 23, 2004.

(51) Int. Cl. A61F 2/28

(52) U.S. Cl. 428/698; 623/23.51; 427/2.26; 148/238; 148/222; 148/317

(57) ABSTRACT

The surface of a medical device is treated by exposure to a gas at a pressure less than atmospheric pressure to produce a hardened, minimally abrasive surface layer substantially comprising chromium nitride.

### CROSS-REFERENCE TO RELATED APPLICATIONS

Not applicable.

### BACKGROUND OF THE INVENTION

The present invention relates generally to medical devices and, more particularly, to a medical device comprising a cobalt-chromium alloy and having a hardened, wear resistant surface of minimal abrasiveness.

To be suitable for use in a medical device, a material must exhibit the appropriate functional properties, mainly mechanical properties, for the particular application and must be biocompatible. Biocompatibility is the ability of a material to perform with an appropriate host response in a particular application. For example, in the complex environment of the human body, metal alloys are subject to electrochemical corrosion with the bodily fluids acting as an electrolyte and, to be biocompatible, a metal alloy used in an implantable medical device must exhibit very low corrosion over the projected lifetime of the device. Metal particles released by corrosion may be concentrated locally or distributed systemically and it is important that the type and amount of material released does not pose a danger to the patient. Cobalt-chromium based alloys, developed for the aerospace industry, are used in many medical device applications, including implantable medical devices, because of their strength, corrosion resistance, and biocompatibility. For example, cobalt-chromium alloys, typified by alloys conforming to ASTM standard specifications, such as, ASTM F-75-01, STANDARD SPECIFICATION FOR COBALT-28 CHROMIUM-6 MOLYBDENUM ALLOY CASTINGS AND CASTING ALLOY FOR SURGICAL IMPLANTS, and ASTM-799, STANDARD SPECIFICATION FOR COBALT-28 CHROMIUM-6 MOLYBDENUM ALLOY FORGINGS FOR SURGICAL IMPLANTS, are often used as components of modular prosthetic devices such as prosthetic hip and knee joints.

A prosthetic joint typically includes paired load bearing surfaces, commonly comprising a first surface of a metal alloy component paired with a second surface comprising a metal, a polymer, a ceramic, bone, or bone cement. When load bearing surfaces move relative to each other, such as during articulation of a prosthetic joint, friction can cause the surfaces to spall. The wear debris, known medically as third bodies, originating from the load bearing surfaces of an implanted medical device can initiate a histiocytic reaction in which the body's immune system is activated to release enzymes to dissolve the particles of debris. However, because the wear debris is usually a relatively hard material, such as a metal or polycarbon compound, the enzymes either fail to dissolve the debris or dissolve the debris only with the passage of considerable time. On the other hand, the enzymes do react with tissue and bone and may weaken or dissolve the bone supporting or adjacent to the medical device. In the case of a prosthetic joint, weakening of the bone or osteolysis may shorten the life of the device and may eventually render the supporting bone unusable. Further, surface erosion can eventually lead to failure of the load-bearing surfaces, requiring replacement or repair of the surfaces. In the case of implanted medical devices, replacement or repair entails expensive and risky surgery.

Cobalt-chromium alloy surfaces can be hardened by several methods to reduce wear. By way of examples, the surface of a cobalt-chromium alloy may be hardened by depositing a titanium nitride coating on the surface, or by ion implantation of the cobalt-chromium matrix. Surface hardening methods also include gas nitriding, chemical salt bath nitriding, plasma or ion nitriding, and ion implantation.

Cobalt-chromium based alloys can also be strengthened by adding nitrogen to the alloy in the molten state or by diffusing nitrogen into the alloy in the solid state. Specifically, forming gas comprising 15% hydrogen and 85% nitrogen, in combination with either ammonia or argon, may be used to diffuse nitrogen into a cobalt-chromium alloy. However, such processes change the chemistry of the alloy by significantly increasing the nitrogen present throughout the alloy and it has been found that when nitrogen is added in this manner, the fracture toughness of the alloy is reduced significantly. Further, hydrogen produced by the dissociation of ammonia can result in hydrogen embrittlement and the process can also cause decarburization, the loss of carbon from the surface of the alloy, which can reduce the hardness of the material.

Shetty et al., U.S. Pat. No. 5,308,412, disclose a method of surface hardening cobalt-chromium based alloys wherein a surface of the alloy is exposed to pure molecular nitrogen gas or ionized nitrogen under a positive pressure at a temperature preferably about 1400° F. for approximately 48 hours. The result is a hardened diffusion layer bounded by the surface and a substrate of cobalt-chromium alloy. This process enhances the hardness and wear resistance of the surface of the cobalt-chromium alloy with minimal reduction in fatigue resistance. According to the disclosure, exposing the surface to pure nitrogen at 1-2 psig. positive pressure and at a temperature in the specified range prevents the formation of a chromium nitride compound layer on the surface which would increase surface roughness and reduce wear resistance.

While surface hardening can substantially reduce wear of a cobalt-chromium load bearing surface, the paired load bearing surface is commonly a dissimilar material, such as a polymer, a ceramic, bone, or bone cement which is also subject to wear. For example, a substantial majority of total hip and knee replacements have incorporated either a conventional or highly cross-linked ultra-high-molecular-weight-polyethylene (UHMWPE or UHMWXLPE) for the second of the paired load bearing surfaces because of the low coefficient of friction between polyethylene and cobalt-chromium surfaces. While hardening the cobalt-chromium load bearing surface protects the surface and reduces the amount of cobalt-chromium wear debris, spalling of the corresponding polyethylene load bearing surface of an articulating joint can generate large numbers of submicron-sized polyethylene wear particles that float in the vicinity of the medical device producing an inflammatory response by the immune system. A number of investigations have been initiated seeking to improve the wear resistance of polyethylene or to find an alternative to polyethylene. However, the combination of polyethylene and cobalt-chromium continues to be a material combination of choice for prosthetic joints and other medical devices and the wear debris produced by relative movement a hardened cobalt-chromium load bearing surface and a paired second load bearing surface of polyethylene or another material remains a significant problem related to implanted medical devices.

What is desired, therefore, is a medical device comprising a cobalt-chromium alloy that has a high strength, wear resistant surface that minimizes erosion of a second, paired, load bearing surface.

### BRIEF DESCRITION OF THE DRAWINGS

FIG. 1 is a schematic diagram depicting a prosthetic hip joint in place.

FIG. 2 is a micrograph of a sample of a casting comprising a cobalt-chromium alloy.

FIG. 3 is a flow diagram of a surface treatment process to create a hardened, wear resistant, minimally abrasive surface on a component comprising a cobalt-chromium alloy.

FIG. 4 is a schematic diagram depicting a prosthetic knee joint in place.

FIG. 5 is a schematic diagram depicting the parts of an exemplary prosthetic knee joint.

FIG. 6 is a micrograph of a sample of a cobalt-chromium alloy having a surface layer substantially comprising chromium nitride.

FIG. 7 is a micrograph of a second sample of a cobalt-chromium alloy having a surface layer substantially comprising chromium nitride.

### DETAILED DESCRIPTION OF THE INVENTION

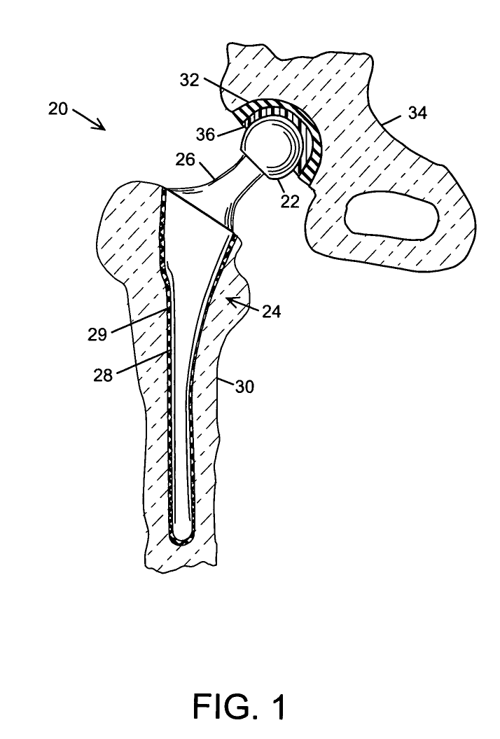

Referring in detail to the drawings where similar parts of the invention are identified by like reference numerals, and, more particularly to FIG. 1, an artificial joint, such as the prosthetic hip joint 20, exemplifies a medical device, and, more specifically, an implantable medical device, that includes a pair of interfacing load bearing surfaces arranged to move relative to each other. The prosthetic hip joint 20 typically includes a ball 22 that is connected to a body 24 comprising a neck 26 and a stem 28. The stem 28 may be held in place in the femur 30 by a variety of methods, including the use of cementing agents 29, an interference fit, a threaded attachment mechanism, or biological fixation.

A cup-shaped socket 32 is anchored in the pelvis 34 by any of a variety of known techniques, such as cementing; press fitting; the use of screws; the use of a textured, knurled, or threaded exterior; the use of a biological fixation mechanism or by a combination of biological and mechanical fixation. The ball 22 is positioned adjacent to the concave surface of the socket 32. A socket insert 36; commonly comprising a polymer, such as an ultra-high molecular weight polyethylene (UHMWPE) or an ultra-high molecular weight, cross linked polyethylene (UHMWXLPE), is disposed within the socket 32 to reduce friction between the ball 22 and the socket and increase the life of the joint. On the other hand, the socket insert 36 may comprise ceramic or metal or a ceramic or metal socket may be used without a socket insert. In some cases, the second load bearing surface in contact with a surface of a medical device component of cobalt-chromium comprises bone or bone cement. The convex outer surface of the ball 22 interfaces with the concave load bearing surface of the socket insert 36 or socket 32, as appropriate, to allow the joint to rotate and articulate simulating the movement of the natural hip joint.

For strength, corrosion resistance, and biocompatibility, the ball 22 comprises a cobalt-chromium alloy. Additional components of the prosthetic hip joint 20, including the body 24 and the socket 32 may also comprise a cobalt chromium alloy. Cobalt-chromium alloys are alloys comprising significant portions of cobalt and chromium and, commonly, also include a significant portion of molybdenum. Cobalt-chromium alloys used in medical devices are typified by alloys complying with ASTM standard specifications, ASTM F-75-01, STANDARD SPECIFICATION FOR COBALT-28 CHROMIUM-6 MOLYBDENUM ALLOY CASTINGS AND CASTING ALLOY FOR SURGICAL IMPLANTS, and ASTM-799, STANDARD SPECIFICATION FOR COBALT-28 CHROMIUM-6 MOLYBDENUM ALLOY FORGINGS FOR SURGICAL IMPLANTS. Cobalt-chromium alloys also include alloys that have higher minor portions of carbon or nitrogen and comply with an ASTM F-75 Modified specification. In addition, as used herein, cobalt-chromium alloys include other proprietary alloys that contain cobalt and chromium and resemble alloys conforming to the ASTM-F75, modified ASTM-F75, and ASTM-799 standard specifications.

Rotation or articulation of the joint 20 produces relative movement of the outer surface of the ball 22 and the inner surface of the socket insert 36 potentially causing spalling of the interfacing load bearing surfaces and the production of wear debris or third bodies. To reduce wear, resulting from the relative movement of the load bearing surfaces of the joint 20, the surface of the metal ball 22 is typically hardened. While hardening the surface of the ball 22 effectively reduces wearing of the ball and the accumulation of metal wear debris originating from the ball, wear of the interfacing load bearing surface, for example the load bearing surface of the socket insert 36, often limits the life of the prosthetic joint and may produce wear debris that is responsible for a histiocytic reaction or other health hazard for the patient.

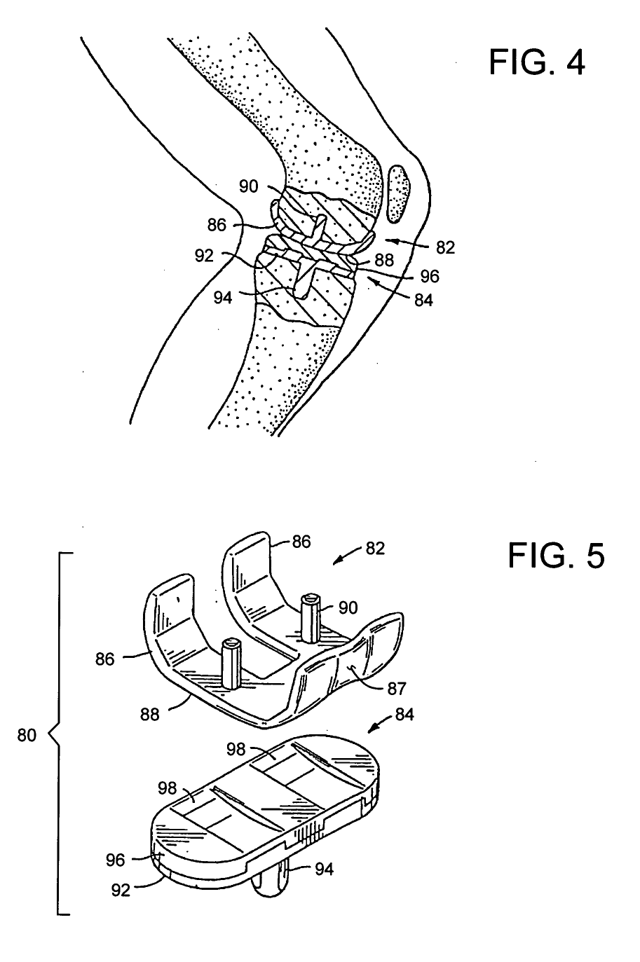

Similarly, a prosthetic knee joint is shown in situ in FIG. 4. Referring also to FIG. 5, the prosthetic knee joint includes a femoral component 82 and a tibial component 84. The femoral component 82 includes a patella track 87 and condyles 86 which include a load bearing surface 88. The femoral component 82 may also include pegs 90 for locating or affixing the femoral component to the femur. The tibial component 84 includes a tibial base 92 with a peg 94 for locating or mounting the tibial base onto the tibia. A tibial platform 96 is mounted atop the tibial base 92 and includes grooves 98 complementary in shape to the condyles 86 of the femoral component 82. The load bearing surfaces 88 of the condyles 86 contact the grooves 98 of tibial platform 96. When the knee flexes, the condyles 86 and the patella track 87 articulate on the tibial platform 96. While the condyles 86 and patella track 87 are typically manufactured of metal, such as a cobalt-chromium alloy, the tibial platform 92 is typically manufactured from a polymer, such as UHMWPE, or a polymer based composite. As in the case of the prosthetic hip joint 20, articulation of the metal load bearing surfaces of the condyles 86 and the patella track 87 against the relatively softer tibial platform 96 may result in wearing of the surfaces of the tibial platform and the production of polymeric wear debris.

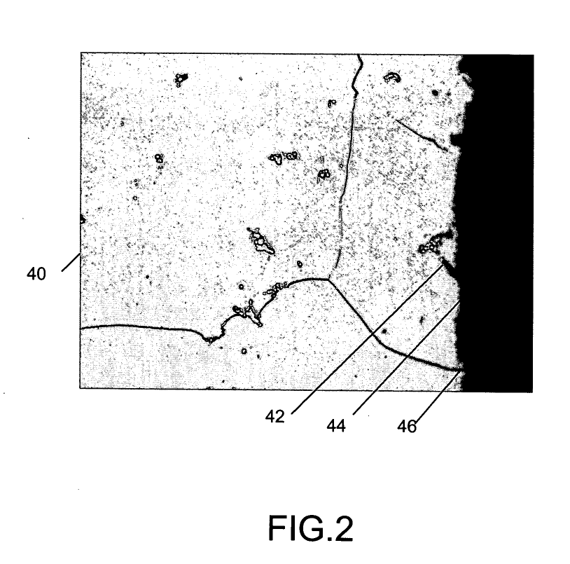

While cobalt-chromium alloys are often used in medical devices because of their strength, corrosion resistance, and biocompatibility, the inventors realized that it is the nature of cobalt-chromium alloys to form isolated particles of carbides of the constituent metals of the alloy. These carbide particles are of random size and are dispersed randomly through the base metal matrix. Referring to FIG. 2, some of the carbide particles 42 form at the surface 44 of the body 40 of the cobalt-chromium component. The inventors realized that the carbide particles are relatively hard compared to the cobalt-chromium base metal. When the surface wears, the softer base metal surrounding the particles erodes leaving the carbide particle standing proud of the surface creating a very abrasive point load on the paired load bearing surface. Attempts to smooth the surface of the cobalt-chromium body by polishing or other mechanical methods are ineffective in reducing the abrasiveness produced by the carbide particles because these processes also remove the softer base metal with little effect on the harder carbides.

The intersection of a boundary 46 between grains of the base metal and a surface 44 of a body 40 is another source of surface abrasiveness. The grain boundary 46 produces a surface discontinuity that is defined by the edges of the adjacent metal grains. While the base material is typically softer than the carbide particles, it is commonly harder than the paired load bearing surface and, with relative movement of the surfaces, the edges of the grains scrape material from the paired load bearing surface.

The inventors came to the unexpected realization that creation of a surface layer substantially comprising chromium nitride could produce a medical device with a very smooth, hardened surface of minimal abrasiveness. The surface layer seals off discontinuities created by the intersections of grain boundaries with the surface and submerges carbide particles at the surface in a smooth layer of biocompatible chromium nitride that is harder than the carbide. The inventors also determined that exposing a surface of a body comprising cobalt and chromium to a reaction gas, including nitrogen, at a pressure less than atmospheric and at a temperature between 250° C. and 1000° C. for a period of time would cause the growth of a smooth compound surface layer comprising substantially chromium nitride.

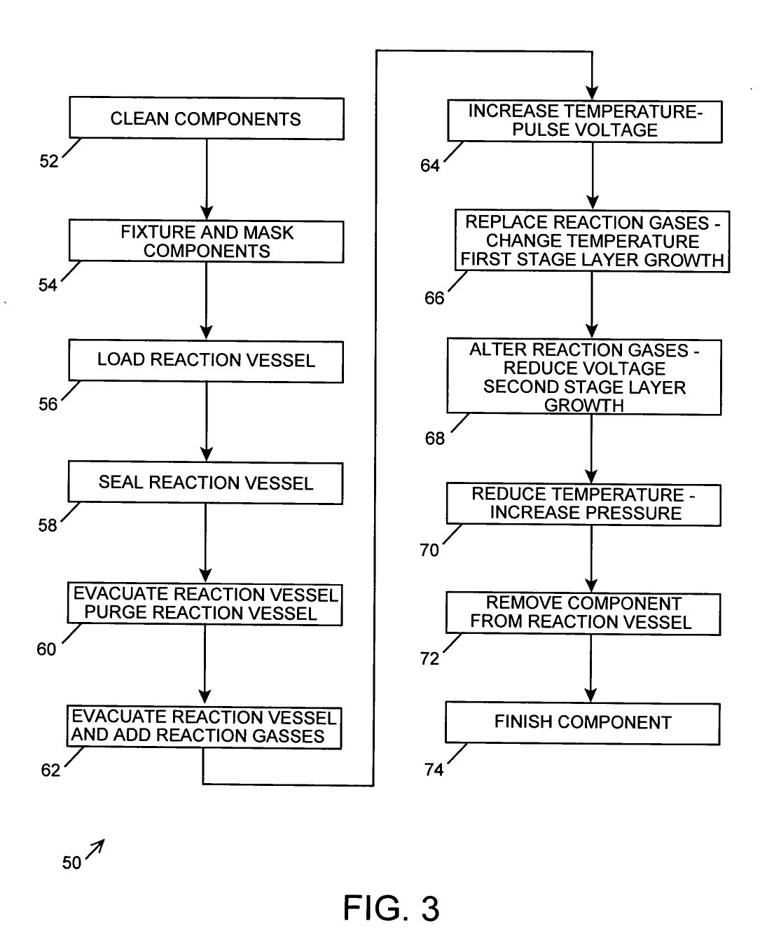

The steps of the surface treatment process 50 for creating a smooth, hardened, minimally abrasive surface for a medical device comprising a cobalt chromium alloy are illustrated in FIG. 3. Commonly, the medical devices to be treated by the process 50 are received for treatment in a fully polished and color buffed condition. Initially, the components are cleaned 52 to remove any foreign material, such as finger prints, finishing or machining oils, polishing or buffing compounds, or other materials encountered during manufacturing, shipping, or handling of the components. Cleaning methods include, but are not limited to, ultrasonic cleaning with and without soaps or surfactants, degreasing with commercially available degreasers, and chemical etching with acids or caustic materials.

Following cleaning, the components are typically loaded into appropriate fixtures 54. The fixtures optimize the exposure of the surfaces to be treated, mask surfaces that are not to be treated, and minimize the opportunity of contact between neighboring components in a batch or load in a reaction vessel. The fixture also promotes equal exposure of all of the components in a load to the process environment in the reaction vessel. The components are loaded into a reaction vessel suitable for plasma nitriding 56 and the reaction vessel is closed and sealed 58.

To promote consistency of results, the environmental gases that entered the reaction vessel during loading are typically purged from the sealed vessel 58. Purging typically comprises the steps of evacuating the reaction vessel to a pressure less than atmospheric and, typically, a pressure less than 1 millibar (mbar), followed by the introduction of nitrogen to force remaining environmental gases out of the reaction vessel. Purging is typically conducted at a temperature between the ambient temperature and 300° C. The specific temperature during purging is determined experimentally and is influenced by the construction of the components to be treated, including the sizes of the components and thicknesses of the various sections of the components, and the sizes and weights of the components making up the load in the reaction vessel. For example, when processing a fully loaded reaction vessel containing a plurality of femoral components of a prosthetic knee, such as the femoral knee component 82, a temperature of approximately 250° C. has been found to be appropriate.

The reaction vessel is evacuated to a pressure less than one atmosphere and then back-filled to a partial vacuum with a reaction gas 62. While the constituency of the reaction gas can be varied for specific components or loads, the reaction gas is typically a mixture of nitrogen, argon, and hydrogen. For example, a mixture of 4-8% nitrogen, 2-4% argon, and 94-98% hydrogen, by volume at a pressure less than one atmosphere, has been found to be suitable for treating a full reaction vessel of femoral knee components 82. In addition, trace amounts of methane may be added to the reaction gas to stabilize the carbon in the treated components. The reaction gas is introduced at a pressure of 2-4 mbar when processing an exemplary load of femoral knee components 82.

Following introduction of the reaction gas, the temperature in the reaction vessel is gradually increased and a pulsing voltage is applied to the reaction gas 64 to clean the exposed surface in preparation for the creation of a chromium nitride surface layer. For example, when processing an exemplary load of femoral knee components, the temperature is raised, at a rate of approximately 150° C. per hour, from approximately 200° C. to 500° C. over a period not less than 2 hours or more than 5 hours. A voltage applied to the reaction gas produces a plasma in the reaction vessel. In the exemplary processing of femoral knee components 82, a pulsed voltage between 450V and 500V with an on-to-off time ratio for the pulse of approximately 2:5 is applied to the reaction gas.

Altering the voltage pulses and the constituency and temperature of the reaction gas 66 initiates a first stage of growth of the compound surface layer. A first stage reaction gas comprising 5-10% nitrogen and, correspondingly, 95-90% hydrogen is introduced to the reaction vessel. The reaction gas, at this step, may also contain small amounts of argon or methane. A partial vacuum with a pressure less than one atmosphere and, preferably, less than 100 mbars is maintained for the reaction gas and the temperature in the reaction vessel is maintained between 450° C. and 600° C. for 3-6 hours. For the exemplary processing of femoral knee components 82, the reaction gas is introduced to the reaction vessel at a flow rate of approximately 150-300 liters per hour with a pressure of 2-4 mbar and the temperature is maintained at 580° C. for approximately four hours. To produce a first stage plasma, the reaction gas is excited with an electrical pulse of, typically, 450-550 volts with an on-to-off time ratio of approximately 1:2.

Following initial growth of the surface layer 66, the reaction gas and the pulse voltage are revised to continue the growth of the surface layer 68 and minimize the diffusion of nitrogen into the component. While the second stage reaction gas continues to comprise 5-10% nitrogen and, correspondingly, 95-90% hydrogen, the concentration of nitrogen in the reaction vessel is increased for the second stage of surface layer growth 68. For example, the percentage of nitrogen in the reaction gas may be increased from 5% for first stage of surface layer growth 66 to 7.5% for second stage of surface layer growth 68. Trace amounts of methane are typically included in the reaction gas during this second stage of surface layer growth to stabilize the carbon in the components. A pressure less than one atmosphere, and preferably less than 100 mbars, is maintained in the reaction vessel during the second stage of surface layer growth 68. In addition, the pulse voltage is reduced to approximately 480 volts for processing the exemplary load of femoral knee components. The components are exposed to the plasma of the second stage reaction gas for 10-20 hours during the second stage of surface layer growth 68. The higher concentration of nitrogen in the reaction gas and lowered pulse voltage produces a second stage plasma that facilitates growth of the compound surface layer while minimizing or avoiding the development of a diffusion layer.

When the components have been exposed to the plasma for sufficient time to permit the surface layer to reach the desired depth, typically 3-15 microns, the temperature in the reaction vessel is reduced and the pressure is increased 70 in preparation for removing the components from the reaction vessel. Over a period of approximately 8 hours, as determined by the size of the load in the reaction vessel, the temperature in the reaction vessel is reduced to approximately 120° C. and the pressure is increased to approximately atmospheric by the introduction of nitrogen gas. The processed components are then removed from the reaction vessel 72 and any final finishing 74, typically limited to polishing for appearance, is performed.

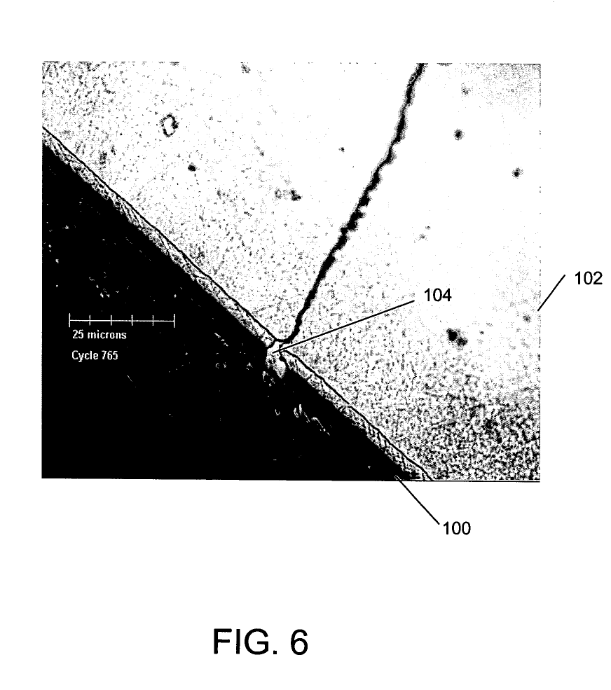

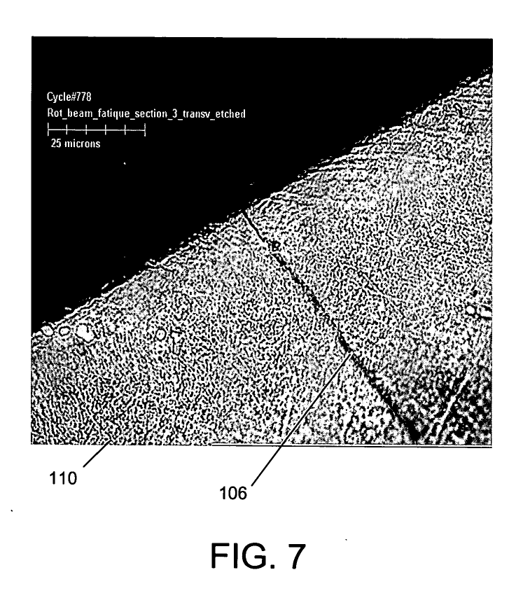

Referring to FIGS. 6 and 7, the surface treatment process 50 causes a compound surface layer 100 to develop at the surface of a body 102 comprising a cobalt-chromium alloy matrix. The surface layer 100 comprises substantially biocompatible chromium nitride. The surface layer is hard and smooth and subsumes particles of carbide, such as the particle 104, at the surface of the body 102 and seals over surface discontinuities, such as those resulting from the intersection of grain boundaries 106 with surface of the body 110. Analysis confirms that components of cobalt-chromium alloy subjected to the surface treatment process 50 develop a 3-15 micron thick compound surface layer substantially comprising chromium nitride that transitions to the cobalt-chromium base metal of the body by way of a transition layer that is much thinner than the surface layer. The result is a medical device with a very smooth, hard, minimally abrasive surface that exhibits a very low coefficient of friction and reduces wear of other materials, such as polyethylene, that are commonly paired with cobalt-chromium load bearing surfaces.

The detailed description, above, sets forth numerous specific details to provide a thorough understanding of the present invention. However, those skilled in the art will appreciate that the present invention may be practiced without these specific details. In other instances, well known methods, procedures, components, and circuitry have not been described in detail to avoid obscuring the present invention.

All the references cited herein are incorporated by reference.

The terms and expressions that have been employed in the foregoing specification are used as terms of description and not of limitation, and there is no intention, in the use of such terms and expressions, of excluding equivalents of the features shown and described or portions thereof, it being recognized that the scope of the invention is defined and limited only by the claims that follow.

1. A medical device having a surface, said surface defining a surface layer substantially comprising chromium nitride.

2. The medical device of claim 1 wherein said surface layer has a depth measured from said surface, said depth being greater than 3 microns.

3. The medical device of claim 1 wherein said surface layer has a depth measured from said surface, said depth being less than 15 microns.

4. The medical device of claim 1 further comprising a transition layer adjacent to said surface layer, said transition layer having a depth less than a depth of said surface layer.

5. The medical device of claim 1 wherein said surface is a load bearing surface.

6. An implantable medical device comprising:
(a) a first surface comprising one of a metal, a polymer, a ceramic, and a bone; and
(b) a body comprising cobalt and chromium, said body having a second surface arranged for movable contact with said first surface, said second surface defining a surface layer substantially comprising chromium nitride.

7. The medical device of claim 6 wherein said surface layer has a depth measured from said second surface, said depth being greater than 3 microns.

8. The medical device of claim 6 wherein said surface layer has a depth measured from said second surface, said depth being less than 15 microns.

9. The medical device of claim 6 further comprising a transition layer adjacent to said surface layer, said transition layer having a depth less than a depth of said surface layer.

10. The implantable medical device of claim 6 wherein said body further comprises molybdenum.

11. The implantable medical device of claim 6 wherein said body comprises an alloy of cobalt and chromium conforming to one of an ASTM-75 standard specification, an ASTM-F-75 Modified standard specification, and an ASTM-799 standard specification.

12. A medical device comprising a body comprising cobalt and chromium, said body including a surface exposed to a gas including nitrogen at a pressure less than one atmosphere and a temperature within a range of 250° C. to 1000° C. for a time sufficient to form a surface layer defined by said surface, said surface layer comprising substantially chromium nitride.

13. The medical device of claim 12 wherein said surface layer has a depth measured from said surface, said depth being greater than 3 microns.

14. The medical device of claim 12 wherein said surface layer has a depth measured from said surface, said depth being less than 15 microns.

15. The medical device of claim 12 further comprising a transition layer adjacent to said surface layer, said transition layer having a depth less than a depth of said surface layer.

16. The medical device of claim 12 wherein said body further comprises molybdenum.

17. The medical device of claim 12 wherein said body comprises an alloy of cobalt and chromium conforming to one of an ASTM-75 standard specification, an ASTM-F-75 Modified standard specification, and an ASTM-799 standard specification.

18. The medical device of claim 12 wherein said gas further comprises at least one of hydrogen, argon, and methane.

19. The medical device of claim 12 wherein said temperature within said range of 250° C. to 1000° C. comprises a temperature within a range of 450° C. to 600° C.

20. The medical device of claim 12 wherein said pressure less than one atmosphere comprises a pressure less than 100 millibars.

21. The medical device of claim 12 wherein said pressure less than one atmosphere comprises a pressure less than 5 millibars.

22. The medical device of claim 21 wherein said pressure less than one atmosphere comprises a pressure greater than 1 millibar.

24. The medical device of claim 12 wherein said time sufficient to form said surface layer comprises a period longer than 8 hours and shorter than 42 hours.

25. A medical device having a surface, said medical device comprising:
(a) a matrix comprising cobalt, chromium and molybdenum; and
(b) a surface layer defined by said surface, said surface layer comprising substantially chromium nitride and having a depth from said surface of at least 3 microns.

26. The medical device of claim 25 wherein, said surface layer is produced by a process comprising the steps of:
(a) exposing said surface to a first stage plasma for a first period of at least two hours, said first stage plasma being produced by exposing a first stage gas comprising less than 10 percent nitrogen and at least 90 percent hydrogen to an electrical pulse having a first stage voltage, said first stage gas having a pressure less than one atmosphere and a temperature within a range of 450° C. to 600° C.; and
(b) exposing said surface to a second stage plasma for a second period, said second period being at least fourteen hours in length and subsequent to said first period, said second stage plasma being produced by exposing a gas comprising a greater percentage of nitrogen than that of said first stage gas to an electrical pulse having a second stage voltage, said second stage voltage being less than said first stage voltage, said second stage gas having a pressure less than one atmosphere and a temperature within a range of 450° C. to 600° C.
